(19) United States
(12) Patent Application Publication
(10) Pub. No.: US 2014/0058192 A1
(43) Pub. Date: Feb. 27, 2014

(54) DEVICE TO MONITOR LIGHT EXPOSURE TO ASSIST SLEEPLESSNESS THERAPY

(71) Applicant: Antoon Johan van Rijn, Summerton, SC (US)

(72) Inventors: Jacob Melchior van Rijn, Summerton, SC (US); Antoon Johan van Rijn, Summerton, SC (US)

(73) Assignee: Antoon Johan van Rijn, Summerton, SC (US)

(21) Appl. No.: 13/969,165

(22) Filed: Aug. 16, 2013

Related U.S. Application Data
(60) Provisional application No. 61/691,457, filed on Aug. 21, 2012.

(51) Int. Cl. A61M 21/00

(52) U.S. Cl. CPC A61M 21/00
USPC 600/26

(57) ABSTRACT

A light monitoring device for treating sleeplessness includes a housing and a light sensing and evaluation system. The light sensing and evaluation system includes a light sensor unit, light measure circuitry measuring light of specific frequencies and intensities, and programmable light evaluation circuitry analyzing the light spectrum exposure for the user based upon measurements made by the light measurement device. The programmable light evaluation circuitry determines when predetermined thresholds relating to exposure of the user to specific light frequencies and intensities are reached. The light monitoring device also includes a warning system providing the user with a warning when the predetermined thresholds are reached and a data storage device storing data generated by the light sensing and evaluation system.

### CROSS REFERENCE TO RELATED APPLICATION

This application claims the benefit of U.S. Provisional Application Ser. No. 61/691,457, entitled “DEVICE TO MONITOR LIGHT EXPOSURE TO ASSIST SLEEPLESSNESS THERAPY,” filed Aug. 21, 2012.

### BACKGROUND OF THE INVENTION

1. Field of the Invention

The invention relates to a light monitoring device and associated system for treating sleeplessness. In particular, the invention relates to a light monitoring device and system for treating sleeplessness by measuring and evaluating exposure to light.

2. Description of the Related Art

Various tools have been developed for the treatment of insomnia and other sleeping disorders. However, and considering the wide range of devices and systems developed to treat insomnia and other sleep disorders, it still remains a problem for many people. As such, the present invention provides a device for assisting those suffering from such sleeping problems in the treatment of insomnia and other sleeping disorders.

### SUMMARY OF THE INVENTION

It is, therefore, an object of the present invention to provide a light monitoring device for treating sleeplessness. The light monitoring device includes a housing and a light sensing and evaluation system. The light sensing and evaluation system includes a light sensor unit, light measure circuitry measuring light of specific frequencies and intensities, and programmable light evaluation circuitry analyzing the light spectrum exposure for the user based upon measurements made by the light measurement device. The programmable light evaluation circuitry determines when predetermined thresholds relating to exposure of the user to specific light frequencies and intensities are reached. The light monitoring device also includes a warning system providing the user with a warning when the predetermined thresholds are reached and a data storage device storing data generated by the light sensing and evaluation system.

It is also an object of the present invention to provide a light monitoring system including a transceiver for wireless communication with a remote system.

It is another object of the present invention to provide a light monitoring system wherein the predetermined thresholds may be adjusted to specific users.

It is a further object of the present invention to provide a light monitoring system wherein the light sensing and evaluation system and the warning system define a data management system supported by the housing.

It is also an object of the present invention to provide a light monitoring system wherein the data management system is built into a circuit board.

It is another object of the present invention to provide a light monitoring system including a battery supported by the housing and powering the light monitoring device.

It is a further object of the present invention to provide a light monitoring system wherein the battery is a rechargeable battery.

It is also an object of the present invention to provide a light monitoring system including a display.

It is another object of the present invention to provide a light monitoring system wherein the housing includes a fastener for attachment to a user.

It is a further object of the present invention to provide a light monitoring system including a switch for automatically activating the light monitoring system.

It is also an object of the present invention to provide a system for treating sleeplessness by measuring and evaluating exposure to light. The system includes a light monitoring device for treating sleeplessness including a housing, a light sensing and evaluation system, a warning system providing the user with a warning when the predetermined thresholds are reached; a transceiver; and a data storage device storing data generated by the light sensing and evaluation system. The system also includes a remote computing device.

It is another object of the present invention to provide a system wherein the remote computing device is a mobile computing device.

It is a further object of the present invention to provide a system wherein the remote computing device is a host computer.

It is also an object of the present invention to provide a system including a remote website portal.

It is another object of the present invention to provide a system wherein the light sensing and evaluation system includes a light sensor unit, light measure circuitry measuring light of specific frequencies and intensities, and programmable light evaluation circuitry analyzing the light spectrum exposure for the user based upon measurements made by the light measurement device, the programmable light evaluation circuitry determining when predetermined thresholds relating to exposure of the user to specific light frequencies and intensities are reached.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a schematic of the present light monitoring device and the associated system.

FIGS. 2 and 3 are respectively a front perspective view and a rear perspective view of a light monitoring device in accordance with the present invention, as well as an associated mobile computing device provide for real-time interaction with the light monitoring device.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

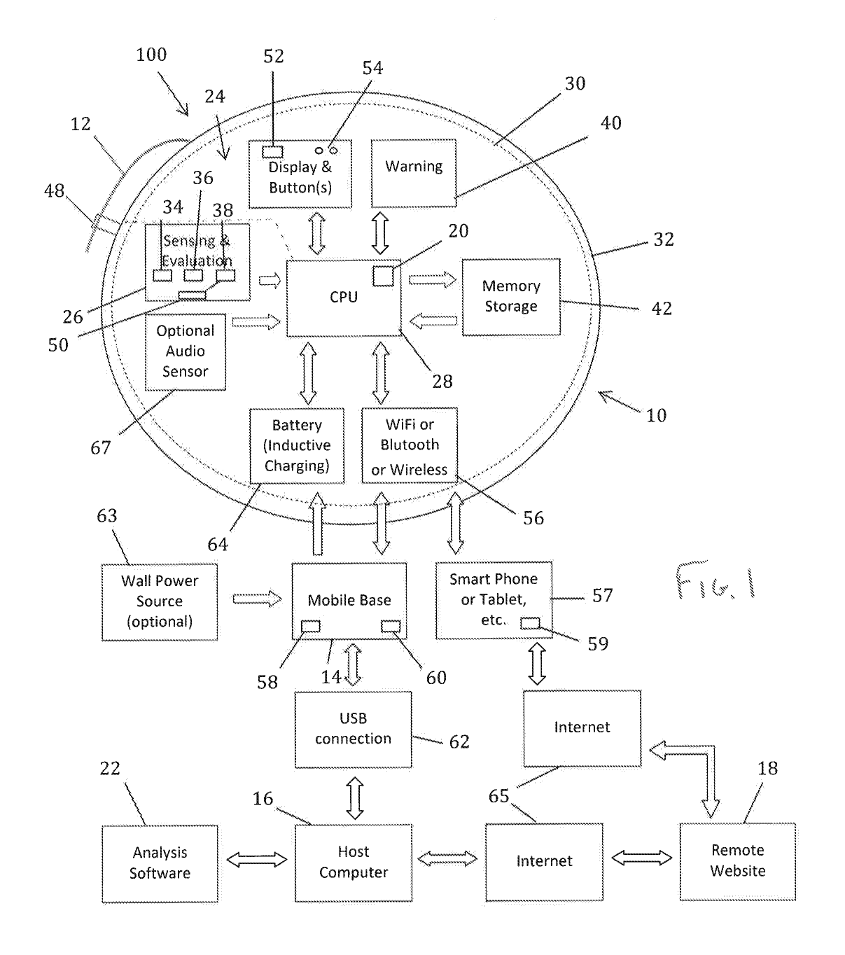

With reference to FIGS. 1, 2 and 3, a wearable light monitoring device 10 for treating sleeplessness in a human by measuring and evaluating exposure to light is disclosed. In particular, a light monitoring device 10 shaped and dimensioned to be worn by a user measures and evaluates light of specific frequencies and intensities during the waking hours by tracking the light spectrum exposure for the wearer (that is, the user) of the light monitoring device 10. Once predetermined thresholds are reached, the light monitoring device 10 warns the wearer that the predetermined levels have been reached thereby allowing the wearer to make any changes necessary to prevent sleeplessness.

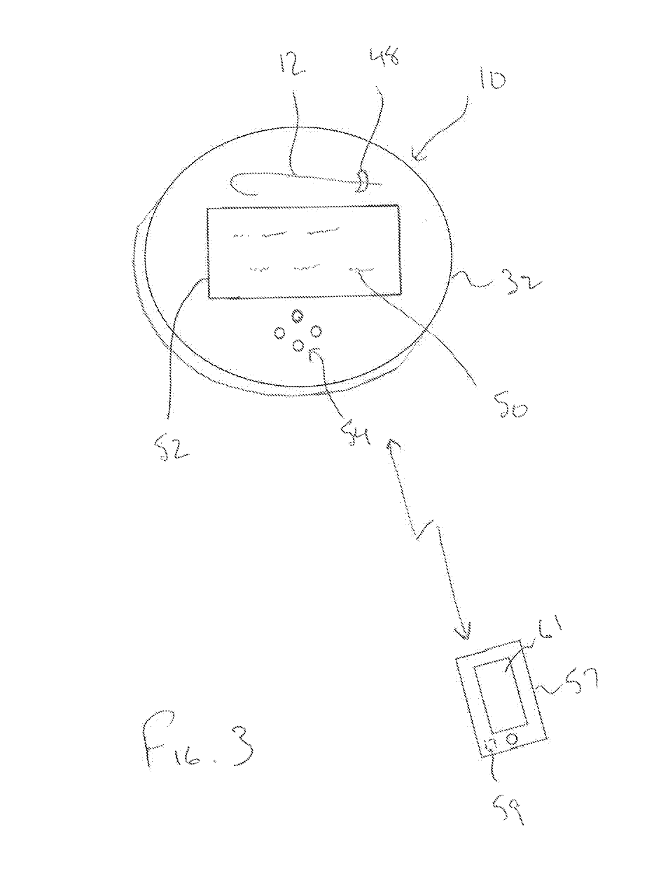

As is explained below in greater detail, and as shown in the schematic of FIG. 1 and the rear perspective view of FIG. 3, the light monitoring device 10 includes a built in fastener 12 allowing for selective and convenient attachment to an article of clothing worn by the wearer. The data generated by the light monitoring device 10 can either be monitored in real time or downloaded through a self contained wireless data link to reveal a complete light exposure pattern and histogram. As such, the light monitoring device 10 may be employed in conjunction with a monitoring system 100 generally composed of the light monitoring device 10, a mobile base unit 14, a remote host computer 16, a remote website portal 18 and/or a mobile computing device 57.

As explained above, predetermined warning levels trigger warnings to the wearer. These trigger levels can be incorporated within the application software 20 of the light monitoring device 10 when certain levels are exceeded, the trigger levels may be incorporated within the analysis software 22 of the remote host computer 16, or the trigger levels may be incorporated within application software 59 of the mobile computing device 57. The application software 20 on the light monitoring device 10, the analysis software 22 of the remote host computer 16, or the application software 59 of the mobile computing device 57 can be personalized to the wearer's individual needs.

The light monitoring device 10 further includes a data management system 24 that contains a light sensing and evaluation system 26, a warning system 40, and a data storage device 42 operating under the control of a microprocessor 28, as well as support hardware. These components are built into a circuit board 30 (that is, the components are implemented in the form of a circuit board) forming an integral part of the light monitoring device 10. As it is desirable for the present light monitoring device 10 to link with remote systems for either data processing or additional data storage, the light monitoring device 10 is further provided with a transceiver 56 for wireless communication with the remote systems as described herein in greater detail. In accordance with a preferred embodiment, the transceiver 56 utilizes industry standard technologies, for example, WiFi, Bluetooth, or other wireless protocols, for the wireless transmission of data.

With the foregoing in mind, and with reference to FIGS. 1, 2 and 3, the light monitoring device 10 for treating sleeplessness is disclosed herein. The light monitoring device 10 includes a housing 32 having a fastener 12 for attachment to a wearer. A plurality of functional components are supported by and integrated within the housing 32. These functional components include a light sensing and evaluation system 26 composed of a light sensor unit 34, light measuring circuitry 36 measuring light of specific frequencies and intensities, programmable light evaluation circuitry 38 analyzing the light spectrum exposure for the wearer based upon the raw measurements made by the light measuring circuitry 36. The functional components also include a warning system 40 providing the wearer with a warning when the predetermined thresholds are reached, and a data (or memory) storage device 42 storing data generated by the light measuring circuitry 36 and the programmable light evaluation circuitry 38.

The housing 32 is conventional in structure and includes walls 44 housing the functional components making up the present light monitoring device 10. These walls 44 include apertures through which various components extend for exposure to the external environment. For example, a light aperture 46 is provided through which the light sensor unit 34 of the light sensing and evaluation system 26 extends for receiving the light spectrum monitored in accordance with the present invention. In accordance with a preferred embodiment, and as will be explained below in greater detail, warnings and alerts are provided via the display. However, it is appreciated warning lights may be integrated into the housing where such lights would add convenience and functionality to the light monitoring device.

As mentioned above, the housing 32 is provided with a fastener 12 for selective attachment to a wearer. The fastener 12 is provided with a switch 48 (in the form of a latch) linked to the data management system 24 of the light monitoring device 10 for automatically providing an indication that the light monitoring device 10 has been attached to/detached from the wearer. For example, when the light monitoring device 10 is attached to the wearer, and the fastener 12 is engaged with the switch 48 (that is, a latch) it is automatically activated and begins the process of measuring and analyzing the light to which the wearer is exposed. Similarly, the interaction between the fastener 12 and switch 48 identifies when the light monitoring device 10 has been removed and shuts down operations.

It accordance with a preferred embodiment, the fastener 12 may take the form of a pin clip for convenient and selective attachment to clothing worn by the wearer. It is, however, appreciated the fastener 12 may take a variety of forms depending upon the specific manner in which the user wishes to secure the housing 32 to his/her clothing or body.

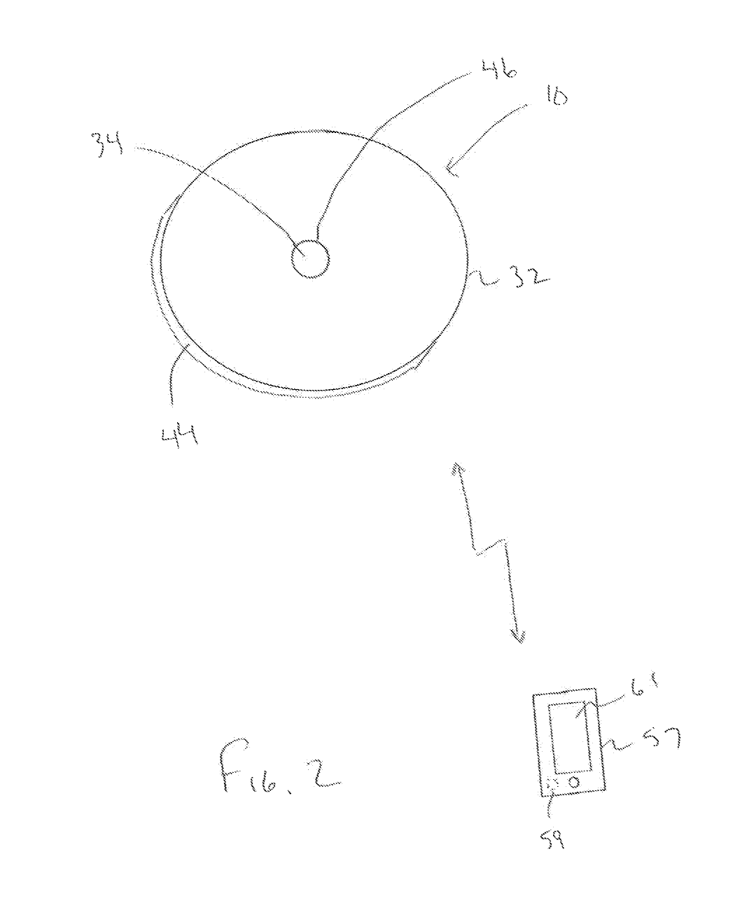

Considering the various possible ways in which it may be desirable for the wearer to secure or carry the present light monitoring device 10, it is appreciated the housing 32 may take a variety of forms. With this in mind, it is appreciated that the light sensor unit should be positioned and oriented so as to have about the same exposure to light as the wearers' eyes. As such, the present light monitoring device 10 is designed for positioning somewhere on the front side of the upper body. For example, the housing 32 may take the form of a wearable button (as shown in FIGS. 2 and 3), customary necklaces with a pendant, a lapel pin, a brooch etc. and may be constructed of plastic, titanium or other lightweight materials. It is contemplated the housing 32 may be round in nature with a diameter of approximately 1.5 to 2 inches, and a thickness of approximately ¼ inch.

The various functional components making up the light monitoring device 10 (that is, the light sensor unit 34, the light measuring circuitry 36, the programmable light evaluation circuitry 38, the warning system 40, and the data storage device 42 and the microprocessor 28, which define a data management system 24) are preferably integrated into a printed circuit board 30 which is mounted within the housing 32. As such, the light sensor unit 34, the light measuring circuitry 36, the programmable light evaluation circuitry 38 and the warning system 40 are housed within the housing 32 and constructed in a convenient one-piece manner.

The light sensor unit 34 is full spectrum RGB light sensor. In accordance with a preferred embodiment, the light sensor unit and the light measuring circuitry may be purchased from existing vendors. For example, the HDJD-S822 Color Sensor manufactured by Avago Technologies may be used. The HDJD-S822 is a high performance, small in size, cost effective light to voltage converting sensor. The sensor combines a photodiode array (that is, the light sensor unit) and three transimpedance amplifiers in a single monolithic CMOS IC solution. With Red (R), Green (G) and Blue (B) color filters coated over the photodiode array, the sensor converts RGB light to analog voltage outputs, denoted by VROUT, VGOUT and VBOUT, respectively. The sensor is packaged in a 5×5×0.75 mm surface mount QFN-16 package. The HDJD-S822 Color Sensor converts light to R, G, B voltage output; includes a monolithic CMOS IC solution comprises of an array of photodiode coated with R, G, B color filter and integrated with trans-impedance amplifier; includes independent gain selection options for each R, G, B channel; and includes uniform photodiode array design to minimize the effect of contamination and optical aperture misalignment.

It is also appreciated the ADJD-S311-CR999 Color Sensor manufactured by Avago Technologies may be used. The ADJD-S311-CR999 Color Sensor offers the advantage of providing customized programming that would allow the ADJD-S311-CR999 Color Sensor to function as the complete light monitoring device 10 and thereby providing the functionality of the light sensor unit 34, the light measuring circuitry 36, the programmable light evaluation circuitry 38.

Regardless of the light sensing and evaluation system 26 employed, the light sensor unit 34 is integrated with a light measuring circuitry 36. The light measuring circuitry 36 gathers data regarding the specific intensities and wavelengths of light to which the wearer is exposed. As such, the light measuring circuitry 36 includes a light frequency filter and intensity monitor to determine the frequencies and intensity of the light to which the wearer is exposed. In accordance with a preferred embodiment of the present invention, the light measuring circuitry 36 includes a three channel sensor array that captures Red, Blue and Green light differences along with light intensity.

The data generated by the light measuring circuitry 36 is employed by the programmable light evaluation circuitry 38. That is, the raw data generated by the light measuring circuitry 36 enumerates raw data into machine ready data that is used for processing in accordance with the present invention and/or sent to a data storage device 42. The raw data processed by the programmable light evaluation circuitry 38 is time stamped and is stored to the data storage device 42 in a timed fashion or when predefined changes to the values from the light sensor are achieved.

Specifically, the programmable light evaluation circuitry 38 analyzes the light spectrum exposure (that is, the specific light frequencies and intensities to which the wearer is exposed) for the wearer based upon the measurements made by the light measuring circuitry 36. The programmable light evaluation circuitry 38 ultimately determines when predetermined thresholds relating to exposure of the wearer to specific light frequencies and intensities are reached.

It is appreciated different users will likely have different response characteristics to light and the light sensing and evaluation system 26, in particular, the programmable light evaluation circuitry 38, is provided with a graphical user interface 50 allowing for adjustment to the predetermined thresholds used in determining when warnings should be provided to the user. That is, the programmable light evaluation circuitry 38 is programmable so as to allow for adjustment of the predetermined thresholds to suit specific users. With this in mind, and considering the various other control options contemplated in accordance with the present light monitoring device 10, the device 10 is provided with a display 52 and interface buttons 54 for visualization of the graphical user interface 50, as well as other display functions. The display 52 and buttons 54 are implemented using various menu screens to control, modify and otherwise utilize the present sleep monitoring system 100. It is further contemplated interfacing with the present light monitoring device 10 may be achieved using touch screen functionality integrating the display 52 with menus allowing for touch activation. It is appreciated the specifics of the graphical user interface 50 employed in accordance with the present invention will follow industry standard programming rules and interfaces, like drop down menus, sliders, radio buttons, or other on screen buttons. As it is desired to minimize the energy needs of the present light monitoring device 10, the screen of any display employed will be a persistent low voltage low power screen. Still further, the display 52 provides visual feedback in conjunction with the warning system 40, which may be a happy or sad type facial expression to indicate levels of blue light exposure historically captured throughout the day.

The light monitoring device 10 is capable of providing real-time analysis of the light spectrum and intensity to which the wearer has been exposed. Similarly, the light monitoring device 10, either by itself or in conjunction with the mobile base unit 14 and the remote host computer 16, may store data for subsequent analysis of data and generation of information relating to the wearer's exposure. With this in mind, a mobile base unit 14 is provided in conjunction with the light monitoring device 10 for the receipt of data from the light monitoring device 10. The mobile base unit 14 is coupled, for example, via a USB connection, to a remote host computer 16 containing analysis software 22 that is considered too extensive for integration on the light monitoring device 10 itself. Evaluation data generated by the remote host computer 16 may then be reviewed on the display of the remote host computer 16 or it may be transmitted back to the light monitoring device 10 for alerting the wearer as deemed appropriate. As is discussed below, access to the data generated by the remote host computer 16 and/or the light monitoring device 10 may also be achieved via mobile computing devices (for example, smart phones, cell phones, tablets, etc.) 57 or a remote website portal 18 using the Internet.

In fact, and considering the transceiver 56 employed by the light monitoring device 10, it is preferred that the light monitoring device 10 will be continuously linked to a smartphone/cell phone 57 carried by the user for expansion of the capabilities offered in accordance with the present invention. With this in mind, the present invention provides a real-time monitoring and feedback option by integrating the light monitoring device 10 with an “app” (that is, the previously mentioned software application 59) running on a mobile computing device 57 (for example, a tablet computing device, smartphone, cell phone, etc.) that signals the light monitoring device 10 to start streaming data to the mobile computing device 57 in real time in order for the mobile app 59 to display directly, on the display 61 of the mobile computing device 57, the levels of received color light. It is appreciated, the mobile app would show by way of indicator dials or bar graph indicators, “Good,” “OK,” or “Bad” levels of Red, Green, Blue light levels as pertaining to parameters set dependant on time of day, for example.

It is appreciated that considering the widespread use of smartphones/cell phones 57, the light monitoring device 10 will most likely be interfacing with such smartphones/cell phones 57 for remote access to data. With this in mind, and considering the display 52 built into the light monitoring device 10 will likely be relatively small, the light monitoring device 10 may interface with a smartphone/cell phone 57 carried by the user. As such, the display of the smartphone/cell phone 57 will receive warnings downloaded from the light monitoring device 10 for display to the user and will also allow for control of the light monitoring device 10 by uploading instructions from the smartphone/cell phone 57 to the light monitoring device 10

The warning system 40 is linked to the light monitoring device 10 and provides the wearer with a warning when the predetermined thresholds are reached. In particular, and as briefly discussed above, the display 52 on the light monitoring device 10 can indicate conditions through iconic type messages. The smartphone/cell phone 57 linked to the light monitoring device 10 then receives information downloaded from the light monitoring device 10 and provides for a more expanded and detailed explanation of the prognosis. It is appreciated that a preferred embodiment does not at this time have tactile or sound feedback, but such features may be incorporated into an application running on the smart phones, cell phones, tablets, etc. linked to the light monitoring device 10.

As substantial data is generated during the operation of the present sleep monitoring system, the data storage device 42 is linked to the light sensing and evaluation system 26, in particular, the programmable light evaluation circuitry 38, for ready access to the generated data. In accordance with a preferred embodiment of the present invention the data storage device 42 is a Flash rewriteable memory module.

Considering the ability to provide for wireless communications, the light monitoring device 10 links to a remote host computer 16 to facilitate adjustment of the predetermined thresholds as discussed; that is, the light sensing and evaluation system 26 of the present light monitoring device 10 is customizable to the user by use of external programming capabilities. Still further, the remote host computer 16 is provided with analysis software 22. In particular, the analysis software 22 resides on the remote host computer 16, and receives data from the light monitoring device 10 for analysis on historical data retention. The analysis software 22 is responsible for communicating data, status and updates to the light monitoring device 10 and contains code for data analysis in order to establish baseline and recommendation prognosis.

The linking of the light monitoring device 10 to a remote computer is facilitated by the provision of a mobile base unit 14. The mobile base unit 14 contains a power control subsystem 58 including a regulated house current adapter for use in charging the light monitoring device 10 when the light monitoring device 10 is physically attached thereto, preferably through the use of USB based charging circuits and/or an optional wall power source 63. The mobile base unit 14 also contains a wireless radio interface subsystem 60 that enables wireless data exchange between the light monitoring device 10 and the mobile base unit 14 based on industry standard like WiFi or Bluetooth technology. Transmission of data received by the mobile base unit 14 to the remote host computer 16 is facilitated by a USB interface 62 that allows data communications between the mobile base unit 14 and the remote host computer 16, although it is also contemplated known wireless transmission protocols may be employed. It is also appreciated that the mobile base unit 14 may include indicators like power/charging status and wireless connectivity.

The wireless communication capabilities of the present invention may be implemented for the transmission of data and information to a health care professional for further analysis and treatment. With this in mind, the system in accordance with the present invention is provided with a remote website portal 18 that may be accessed via the Internet 65. The remote website portal 18 processes and stores data received and transmitted throughout the complete system 100. The remote website portal 18 allows for both client login and professional healthcare provider login, wherein client login allows for review and access to status information for personal use and the professional healthcare provider login allows for remote monitoring and prognosis recommendation management.

It is further contemplated the light monitoring device 10 may be provided with an audio sensor 67. Such as sensor might be implemented to monitor sound levels to which the wearer is exposed and which might having a bearing on the sleep habits of the user.

The light monitoring device 10 is further provided with a battery 64 supported by the housing 32. The battery 64 is used to power the light monitoring device 10. In accordance with a preferred embodiment, the battery 64 is a rechargeable battery. It is appreciated a solar or photovoltaic element may be employed to assist in charging the battery 64. The light monitoring device 10 can be worn as jewelry wherein the photovoltaic element is hidden behind an ornate object. Moreover, an induction charging system can be incorporated so that the device is completely sealed without any external connectors. As such, and within current and existing technology, the housing 32 can be constructed as a water resistant enclosure.

As discussed above, the present invention provides a device for assisting users in the treatment of insomnia and other sleeping disorders. Still further, it is contemplated the present invention may be employed by soldiers deployed to foreign lands as a tool to assist in the treatment of sleep disorders. For example, soldiers may be equipped with the present light monitoring device 10 to determine differences in ambient light at their deployed locations when compared to their homes. For example, the exposure to TVs, computers, fluorescent lights, etc. is likely very different from the environment soldiers will encounter in the field, and proactively addressing these differences may lead to enhanced sleep and improved functioning while awake.

Still further, it is also known that lack of sleep for the first few hours of the night can cause a very deep sleep in the second part of the night and sleep apnea is a symptom during deep sleep. As such, the present light monitoring device 10 may be used as a tool to enhance sleep during the first few hours of the night so as to avoid the deep sleep associated with sleep apnea.

While the preferred embodiments have been shown and described, it will be understood that there is no intent to limit the invention by such disclosure, but rather, is intended to cover all modifications and alternate constructions falling within the spirit and scope of the invention.

1. A light monitoring device for treating sleeplessness, comprising:
a housing;
a light sensing and evaluation system comprising a light sensor unit, light measure circuitry measuring light of specific frequencies and intensities, and programmable light evaluation circuitry analyzing the light spectrum exposure for a user based upon measurements made by the light measurement device, the programmable light evaluation circuitry determining when predetermined thresholds relating to exposure of the user to specific light frequencies and intensities are reached;
a warning system providing the user with a warning when the predetermined thresholds are reached; and
a data storage device storing data generated by the light sensing and evaluation system.

2. The light monitoring device according to claim 1, further including a transceiver for wireless communication with a remote system.

3. The light monitoring device according to claim 1, wherein the predetermined thresholds may be adjusted to specific users.

4. The light monitoring device according to claim 1, wherein the light sensing and evaluation system and the warning system define a data management system supported by the housing.

5. The light monitoring device according to claim 4, wherein the data management system is built into a circuit board.

6. The light monitoring device according to claim 1, further including a battery supported by the housing and powering the light monitoring device.

7. The light monitoring device according to claim 6, wherein the battery is a rechargeable battery.

8. The light monitoring device according to claim 1, further including a display.

9. The light monitoring device according to claim 1, wherein the housing includes a fastener for attachment to a user.

10. The light monitoring system according to claim 1, further including a switch for automatically activating the light monitoring system.

11. A system for treating sleeplessness by measuring and evaluating exposure to light, comprising:
a light monitoring device for treating sleeplessness including a housing, a light sensing and evaluation system, a warning system providing a user with a warning when the predetermined thresholds are reached; a transceiver; and a data storage device storing data generated by the light sensing and evaluation system;
a remote computing device.

12. The system according to claim 11, wherein the remote computing device is a mobile computing device.

13. The system according to claim 11, wherein the remote computing device is a host computer.

14. The system according to claim 11, further including a remote website portal.

15. The system according to claim 11, wherein the light sensing and evaluation system includes a light sensor unit, light measure circuitry measuring light of specific frequencies and intensities, and programmable light evaluation circuitry analyzing the light spectrum exposure for the user based upon measurements made by the light measurement device, the programmable light evaluation circuitry determining when predetermined thresholds relating to exposure of the user to specific light frequencies and intensities are reached.

16. The system according to claim 15, wherein the predetermined thresholds may be adjusted to specific users.

17. The system according to claim 15, wherein the light sensing and evaluation system and the warning system define a data management system supported by the housing.

18. The system according to claim 15, further including a battery supported by the housing and powering the light monitoring device.

19. The system according to claim 18, wherein the battery is a rechargeable battery.

20. The system according to claim 15, wherein the light monitoring device includes a display.
